(19) United States
(12) Patent Application Publication
(10) Pub. No.: US 2004/0265389 A1
(43) Pub. Date: Dec. 30, 2004

(54) Degradable gel and method for producing the same

(76) Inventors: Nobuhiko Yui, Ishikawa (JP); Tooru Ooya, Ishikawa (JP); Tuyoshi Nakama, Ishikawa (JP); Ikuo Sato, Kanagawa (JP); Ryouji Kawabata, Kanagawa (JP)

Correspondence Address: WENDEROTH, LIND & PONACK, L.L.P., 2033 K STREET N. W., SUITE 800, WASHINGTON, DC, 20006-1021, US

(21) Appl. No.: 10/829,242

(22) Filed: Apr. 22, 2004

(30) Foreign Application Priority Data
Apr. 25, 2003 (JP) 2003-122860

(51) Int. Cl. A61K 31/728; A61K 9/14

(52) U.S. Cl. 424/488; 514/54; 536/53

(57) ABSTRACT

To provide degradable gel hardly degradable and having enough strength, and a method for producing the same.

Degradable gel with 98% by weight or lower saturated moisture content can solve the problem.

### FIELD OF INVENTION

The present invention relates to degradable gel and its production method.

### BACKGROUND OF THE INVENTION

Materials having degradability are used in wide areas such as medical, food, cosmetic and agricultural fields. Among them, anionic polysaccharides represented by hyaluronic acid mostly possess biodegradability. Hence gel comprising them as main components is expected to be utilized in sustained release of various functional materials.

Production processes of crosslinked products of these anionic polysaccharides with crosslinking agents such as ethyleneglycol diglycidylether and divinylsulfone are publicly known. (refer e.g. to Patent document 1; Patent document 2)

However, these crosslinked products are fragile because their saturated moisture contents are high as 99% by weight. Particularly when used in vivo, there is a problem that they are easily decomposed by active oxygen or oxygen in the living body. Besides the gel comprising polysaccharides as main components, there are gel of collagen and gelatin. However, since the outbreak of bovine spongiform encephalopathy (BSE), animal-derived materials are inclinable to be avoided. Furthermore, the gel of collagen or gelatin occasionally causes inflammation when introduced in the living body.

### PRIOR ART

Patent document 1: Japan patent laid-open Hei-5-229934

Patent document 2: U.S. Pat. No. 4,605,691

### PROBLEMS TO BE SOLVED BY THE INVENTION

The problem for the present invention is to provide degradable gel not easily decomposed and having enough strength, and its production process.

### MEANS FOR SOLVING THE PROBLEM

The present inventors have made diligent researches in consideration of the above problems. Consequently it is found out that the saturated moisture content of the gel can be drastically lowered by dissolving the degradable gel raw material into an aqueous solvent in high concentration and at low viscosity, and crosslinking with a crosslinking agent, and that the thus obtained degradable gel is not decomposed easily and possesses enough strength. The present invention is accomplished based on the above knowledge.

The present invention is constituted as follows:

[1] Degradable gel whose saturated moisture content is 98% by weight or below.

[2] Degradable gel as described in the item [1] in which the degradable gel is polysaccharide gel.

[3] Degradable gel as described in the item [2] in which the polysaccharide gel is anionic polysaccharide gel.

[4] Degradable gel as described in the item [3] in which the anionic polysaccharide gel is hyaluronic acid gel.

[5] Degradable gel as described in the item [1] in which the degradable gel is gel obtained by a crosslinking reaction using a crosslinking agent.

[6] Degradable gel as described in the item [5] in which the crosslinking agent is an epoxy compound having two or more epoxy groups per molecule.

[7] Degradable gel as described in the item [6] in which the epoxy compound is ethyleneglycol diglycidylether.

[8] A method for producing degradable gel having a saturated moisture content of 98% by weight or lower characterized by comprising the following steps:

a first step of preparing a raw material solution of the degradable gel by dissolving the raw material compounds of the degradable gel in an aqueous solvent so as to be 20 to 80% by weight in weight ratio; and

a second step of adding a crosslinking agent to the raw material solution of the degradable gel and crosslinks the raw material of the degradable gel.

### EMBODIMENT OF THE PRESENT INVENTION

The degradable gel of the present invention is characterized by having a saturated moisture content not more than 98% by weight. It is particularly preferable that the degradable gel is crosslinked through crosslinking reaction using a crosslinking agent. The saturated moisture content of the degradable gel of the present invention is preferably not more than 96% by weight, more preferably not more than 93% by weight, furthermore preferably not more than 89% by weight. The lower limit of the saturated moisture content is not particularly specified, however, preferably 50% by weight or higher, more preferably 60% by weight or higher, furthermore preferably 70% by weight or higher, especially preferably 80% by weight or higher. The saturated moisture content in the present invention means a percentage of water in the gel to be calculated by the formula: (mass of wet gel—mass of dry gel)/mass of wet gel×100. Also the mass of the wet gel means a mass in the state reaching equilibrium in pure water at 25° C. Here the state reaching equilibrium means a state of the wet gel left standing in pure water for 100 hours.

The degradable gel means gel having a degrading property under wet environment such as in the living body which is constituted from a polymer compound degrading under such environment and a crosslinking agent, or which decomposes at the bonding sites between the polymer compound and the crosslinking agent. Polymer compounds degrading under such environment and usable as raw materials for the degradable gel of the present invention are illustrated as anionic polysaccharides, cationic polysaccharides, dextran, chitosan, ribonucleic acids, deoxyribonucleic acids, etc. Anionic polysaccharides are particularly preferable for the present invention. The degradable gel used in the present invention may be constituted from a plurality of polymer compounds. Even in the case where a crosslinking agent is used, more than two polymer compounds may be used.

The anionic polysaccharide is a polysaccharide with negative charge due to a carboxyl group, a sulfuric group or the like contained therein, including salts thereof. Concretely the anionic polysaccharide is illustrated as carboxymethyl cellulose, cellouronic acid, alginic acid, alginate, polygalacturonic acid, polygalaturonate, glycosaminoglycan, etc.

The glycosaminoglycan is illustrated as heparin, heparan sulfate, dermatan sulfate, chondroitin sulfate, chondroitin, hyaluronic acid and its salt, etc. These can be utilized singly or as mixture thereof. In the present invention, glycosaminoglycans are preferably used and, among them, hyaluronic acid or its salt (hereafter occasionally called as “hyaluronic acid (salt)”) is especially preferably used.

When the hyaluronic acid (salt) is used as a raw material compound of the degradable gel in the present invention, the average molecular weight of the hyaluronic acid (salt) measured by the HPLC method is preferably not higher than 500 kDa, more preferably not higher than 300 kDa. When the average molecular weight is within the range, gel with a low saturated moisture content can be suitably obtained by conducting crosslinking under the following conditions.

The crosslinking agent usable in the present invention is illustrated as epoxy compounds having two or more epoxy groups per molecule such as ethyleneglycol diglycidyl ether, polyethyleneglycol diglycidyl ether, epichlorohydrin, trimethyl olpropane polyglycidyl ether, neopentylglycol diglycidylether, glycerol polyglycidyl ether, polypropyleneglycol diglycidyl ether, sorbitol polyglycidyl ether, etc., aldehydes having two or more aldehyde groups per molecule such as glutaraldehyde, terephthalaldehyde, etc., polyhydric alcohols such as ethylene glycol, propylene glycol. Among them, an epoxy compound having two or more epoxy groups per molecule, particularly ethyleneglycol diglycidyl ether, can be preferably used. The amount of the crosslinking agent may be preferably 0.01 to 10 equivalents, more preferably 0.05 to 5 equivalents to a functional group reacting with a crosslinking agent.

Production process of the degradable gel of the present invention with a saturated moisture content of not more than 98% by weight comprises the following two processes if broadly divided.

The first process is for preparing a raw material solution of the degradable gel by dissolving the raw material compounds of the degradable gel in an aqueous solvent in the range of 20 to 80% by weight. The second process is for crosslinking the raw material of the degradable gel by adding a crosslinking agent to the raw material solution of the degradable gel.

For gelling an anionic polysaccharide as the raw material of the degradable gel, crosslinking may be carried out after dissolving the anionic polysaccharide in an aqueous solvent. For the aqueous solvent, an alkaline aqueous solution can be used, concentration of which is not specifically limited as long as it permits full advancement of crosslinking by an epoxy compound having two or more epoxy groups per molecule. In case of a sodium hydroxide aqueous solution, for example, its concentration is preferably 0.01 to 10 mol/L, more preferably 0.1 to 5 mol/L. For dissolving hyaluronic acid (salt) in the sodium hydroxide aqueous solution of the above concentration, the concentration of hyaluronic acid (salt) in the aqueous solution is preferably 20% by weight or higher, more preferably 20 to 50% by weight, furthermore preferably 30 to 50% by weight. By adjusting the concentration of hyaluronic acid (salt), viscosity of the obtained hyaluronic acid (salt) solution may be controlled to 5×10⁴ mPas or lower, preferably 1×10⁴ mPas or lower, more preferably 5×10³ mPas or lower. When an anionic polysaccharide is dissolved in a solvent at a concentration of 20% by weight or more, viscosity of the solution is 2×10⁴ mPas or lower. When viscosity is 5×10⁴ mPas or lower, uniform gel can be obtained because defoaming is facilitated and a crosslinking agent can be mixed uniformly.

A production process of the degradable gel is shown below by illustrating the production of hyaluronic acid-epoxy compound gel: Hyaluronic acid having an average molecular weight of 500 kDa or less, preferably 300 kDa or less is dissolved in a sodium hydroxide aqueous solution of 0.01 to 10 mol/L, preferably 0.1 to 5 mol/L concentration, so that the concentration of the hyaluronic acid becomes 20 to 80% by weight, preferably 30 to 50% by weight. The obtained viscous solution is deaerated by using an aspirator. An epoxy compound having two or more epoxy groups per molecule, preferably ethyleneglycol diglycidyl ether, is added by 0.1 to 10 equivalents, preferably 0.5 to 5 equivalents to the disaccharide unit to the solution and stirred. Casting this solution immediately into the prescribed mold, warming in a thermostatic chamber, and forming gel through crosslinking reaction. Temperature of the thermostatic chamber is 50 to 100° C., preferably 60 to 80° C. Time for warming is 10 min or longer, preferably 10 min to 24 hours. The gel taken out from the mold is fully neutralized by an acid solution such as hydrochloric acid aqueous solution, and washed and replaced by distilled water or the equivalent pure water, which is meant by the water purified with electric deionization, reverse osmosis or the like. If necessary, it is replaced by a phosphate buffer, physiological saline or the like for use.

The application of the degradable gel of the present invention is not specifically limited but usable as various materials to be used in medical field, food field, cosmetic field, etc. When the degradable gel is also in vivo degradable, decomposition rate in the living body can be controlled by selecting a saturated moisture content, and the gel is metabolized in the living body, therefore it can be used in the medical field as a humectant at the operation, a lubricant, a wound dressing, a DDS (drug delivery system) material or the like, for example. Particularly since its decomposition rate in a uterus is considered to show high correlation with biorhythm in the human body (uterus), it can be highly effectively utilized as a device for in-uterus or in-vagina implant preparation carrying drugs for endometriosis, for example.

### EXAMPLES

The present invention is described in detail by the following.

1) Measurement of an Average Molecular Weight by HPLC

Although any column can be used which is suitable for the measurement of molecular weight of polysaccharides, it is preferable, if the polysaccharide is hyaluronic acid (salt), to use a column of, for example, Shodex Ionpak KS806 (trade name) or Shodex lonpak KS-G (trade name) both made by SHOWA DENKO K. K. or the like. In Examples and Comparative examples of the present invention, Shodex lonpak KS806 (trade name) and Shodex lonpak KS-G (trade name) both made by SHOWA DENKO K. K are used. In this case, 0.2 mol/L sodium chloride aqueous solution is used as the eluate and flown at the flow rate of 1.0 mL/min. Detection of hyaluronic acid (salt) is made at a wavelength of 206 nm. The average molecular weight can be obtained by calculation using a calibration line made for sodium hyaluronate of known molecular weight obtained by intrinsic viscosity.

2) Measurement of Viscosity

Viscosity of the degradable gel raw material solution can be measured by a rotary viscometer. In Examples and Comparative Examples of the present invention, VISCONIC EHD (trade name) made by TOKIMEC INC. is used and measurement is carried out at the conditions of 25° C. temperature and 0.5 to 100 rpm number of rotation.

Production of hyaluronic acid-ethyleneglycol diglycidyl ether gel (hereafter occasionally called as “CHA-EGDGE gel”) is illustrated as follows:

### Examples 1 to 5 and Comparative Examples 1 and

1. Production of CHA-EGDGE Gel

Sodium hyaluronate (CHA made by CHISSO Co., hereafter occasionally called as “CHA”) is dissolved in 1 mol/L sodium hydroxide aqueous solution by stirring with a spatula and defoamed by an aspirator. A mixed solution of ethyleneglycol diglycidyl ether (Quetol 651 (trade name) made by WAKO PURE CHEMICAL INDUSTRIES, Ltd., hereafter occasionally called as “EGDGE”) and ethanol is added to this with stirring. The obtained viscous solution is immediately cast into a mold having 15 mm diameter and 2 mm depth made of Teflon (registered trade mark), covered with a slide glass plate, and warmed at 80° C. in a thermostatic chamber. The obtained gel is neutralized with 50% by weight ethanol aqueous solution containing 0.05 mol/L hydrochloric acid aqueous solution for one hour, further washed with 50% by weight ethanol aqueous solution and replaced by pure water to obtain gel (hereafter the gel is occasionally called as “HA-EGDGE gel”). The charged amount and molecular weight of CHA, the volume of sodium hydroxide aqueous solution, the charged amount of EGDGE and ethanol, the warming time and the saturated moisture content are shown in Table 1. Uniform and transparent gel is obtained in Examples 1 to 5 and Comparative Examples 1 and 2. Results of viscosity measurement for CHA in 1 mol/L sodium hydroxide solution are shown in Table 2.

2. Degradation Experiments of CHA-EGDGE Gel with Hyaluronidase

CHA-EGDGE gel (Examples 1 to 4, Comparative Examples 1 and 2) is immersed in 25 mL phosphate buffer solution (pH 4.5; 0.14 mol/L) containing 10 unit/mL hyaluronidase (bovine orchis-derived, Type IV-S made by SIGMA-ALDRICH Co.). The hyaluronidase-containing phosphate buffer solution is periodically replaced and the variation in weight of CHA-EGDGE gel is measured. FIG. 1 shows accumulated values of degraded amount of gel plotted with time. It can be seen that the degradation proceeds linearly with time which indicates that the degradation of the gel took place from the surface. FIG. 2 shows the relation between the linear rate of degradation of gel and the saturated moisture content which indicates that there is correlation like an exponential function. Here, the rate of degradation can be drastically reduced by lowering the saturated moisture content. Namely the linear rates of degradation in Examples 2 to 5 is around 1/3 to 1/100 against Comparative Example 1 or 2, and in Example 1, degradation did not take place even after four months. Also gel for Examples 1 to 5 is far sturdier than for Comparative Examples 1 and 2, and easy to be handled without fracturing unexpectedly.

3. Degradation Experiments of CHA-EGDGE Gel with Active Oxygen (Hydroxyl Radical)

Each sample of CHA-EGDGE gel (Example 1 to 4) was immersed in 50 mmol/L ferrous sulfate solution for two days, then immersed in 5 mmol/L hydrogen peroxide aqueous solution and shaken at 25° C. Linear rate of degradation was obtained by measuring the weight periodically. FIG. 3 is a plot of the relation between the linear rate of degradation and the saturated moisture content. It is understood that the rate of degradation can be drastically decreased by lowering the saturated moisture content as there exists a correlation like an exponential function similar to the case of enzymatic degradation.

TABLE 1

| | CHA | | | | | | | |
|---|---|---|---|---|---|---|---|---|
| | Mol | CHA | NaOH | | Eth- | Warm | Warm | SMC |
| | Wt | Feed | Aq.soln | EGDGE | anol | Temp | Time | (note) |
| | (kDa) | (g) | (mL) | (mg) | (mL) | (° C.) | (min) | (%) |
| Ex. 1 | 90 | 1.5 | 3.5 | 870 | 0 | 80 | 20 | 86 |
| Ex. 2 | 90 | 1.5 | 3.5 | 870 | 0 | 80 | 15 | 89 |
| Ex. 3 | 90 | 1.5 | 3.5 | 870 | 0 | 80 | 14 | 94 |
| Ex. 4 | 90 | 1.5 | 3.5 | 870 | 0 | 80 | 12 | 98 |
| Ex. 5 | 230 | 1.5 | 3.5 | 870 | 0.1 | 80 | 15 | 93 |
| C. | 1000 | 0.75 | 4.25 | 435 | 0.1 | 80 | 15 | 99.6 |
| Ex. 1 | | | | | | | | |
| C. | 1000 | 0.75 | 4.25 | 435 | 0.1 | 80 | 30 | 99.5 |
| Ex. 2 | | | | | | | | |

Note:
SMC means saturated moisture content

TABLE 2

| CHA | | | |
|---|---|---|---|
| concentration | Molecular weight (kDa) | | |
| (wt. %) | 90 | 230 | 1000 |
| 15 (rpm) | | 287 mPas | 26500 mPas |
| | | (50 rpm) | (1.0 rpm) |
| 30 (rpm) | 957 mPas | 9260 mPas | |
| | (50 rpm) | (2.5 rpm) | |

### Effects of the Present Invention

Through using the degradable gel of the present invention, time for degradation by an enzyme is drastically extended and sturdier gel than conventional one can be obtained. Thus industrial application range for the gel is widened.

### BRIEF DESCRIPTION OF FIGURES

FIG. 1 A graph illustrating change of accumulated degradation with time for CHA-EGDGE gel.

FIG. 2 A graph illustrating the relationship between the linear rate of degradation of CHA-EGDGE gel and the saturated moisture content.

FIG. 3 A graph illustrating the relationship between the linear rate of degradation of CHA-EGDGE gel and the saturated moisture content in the degradation experiment by the active oxygen

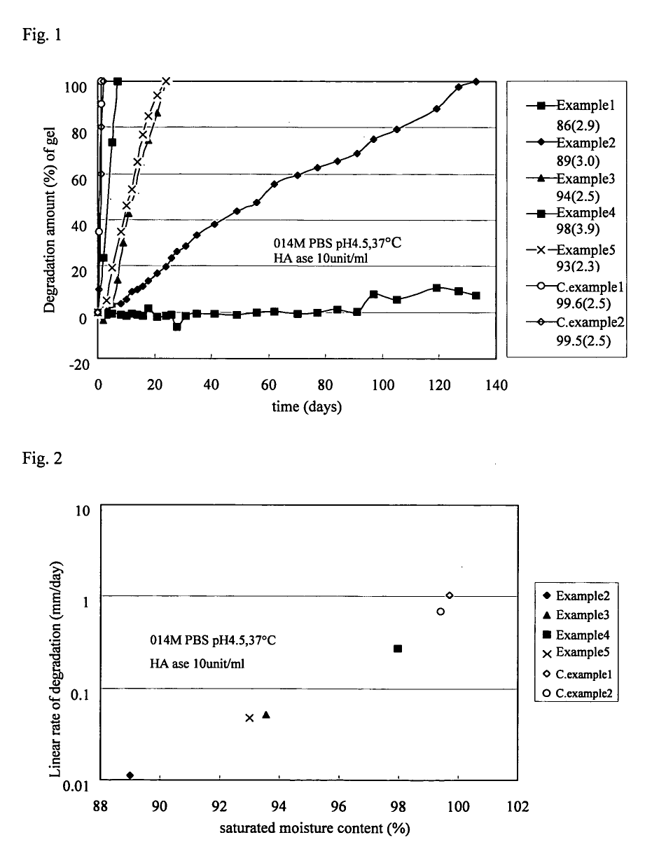

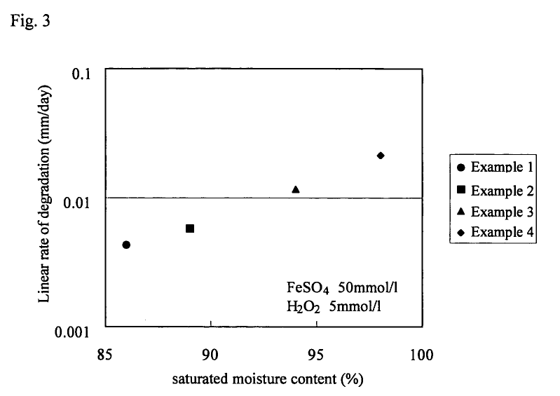

1 Degradable gel whose saturated moisture content is 98% by weight or below.

2 Degradable gel according to claim 1 in which the degradable gel is a polysaccharide gel.

3 Degradable gel according to claim 2 in which the polysaccharide gel is anionic polysaccharide gel.

4 Degradable gel according to claim 3 in which the anionic polysaccharide gel is hyaluronic acid gel.

5 Degradable gel according to claims 1 in which the degradable gel is gel obtained by a crosslinking reaction using a crosslinking agent.

6 Degradable gel according to claim 5 in which the crosslinking agent is an epoxy compound having two or more epoxy groups per molecule.

7 Degradable gel according to claim 6 in which the epoxy compound is ethyleneglycol diglycidylether.

8 A method for producing degradable gel having a saturated moisture content of 98% by weight or lower characterized by comprising the following steps:
a first step of preparing a raw material solution of the degradable gel by dissolving the raw material compounds of the degradable gel in an aqueous solvent so as to be 20 to 80% by weight in weight ratio; and
a second step of adding a crosslinking agent to the raw material solution of the degradable gel and crosslinks the raw material of the degradable gel.
